(19) United States
(12) Patent Application Publication
(10) Pub. No.: US 2019/0076294 A1
(43) Pub. Date: Mar. 14, 2019

(54) COOLING SLEEVE AND TOURNIQUET

(71) Applicant: University of Washington, Seattle, WA (US)

(72) Inventor: Shahram Aarabi, Seattle, WA (US)

(73) Assignee: University of Washington, Seattle, WA (US)

(21) Appl. No.: 16/084,896

(22) PCT Filed: Mar. 15, 2017
(86) PCT No.: PCT/US17/22442

§ 371 Date: Sep. 13, 2018

Related U.S. Application Data
(60) Provisional application No. 62/308,425, filed on Mar. 15, 2016.

(51) Int. Cl. A61F 7/02; A61B 5/01; A61B 5/026

(52) U.S. Cl. CPC A61F 7/02; A61F 2007/0039; A61B 5/026; A61B 5/01

(57) ABSTRACT

A cooling tourniquet system (100) includes a cooling component (110) with a tourniquet (112) at a proximal end and a control component (130). The cooling component includes a cooling pad (120) configured to be wrapped about an extremity, and a cover (111) that wraps around the cooling pad. An inner surface of the cooling pad includes one or more sensors for detecting the temperature of the extremity. The tourniquet is configured to be applied to the extremity to stop blood flow distally from the tourniquet. The control component receives temperature data from the sensors, and controls the cooling pad based on the received data rapidly cooling the extremity at depth, without causing frostbite tissue damage. In one embodiment the control component circulates a chilled fluid with a time-varying temperature profile through passages in the cooling pad. In an embodiment the cover includes an air bladder that holds the cooling pad in contact against the extremity.

### CROSS-REFERENCE TO RELATED APPLICATION

This application claims the benefit of Provisional Application No. 62/308,425 filed Mar. 15, 2016, the entire disclosure of which is hereby incorporated by reference herein.

### BACKGROUND

Acute limb ischemia (ALI) is sudden interruption of limb perfusion and can be caused by limb trauma, atherosclerosis, or life-saving hemostatic therapies such as tourniquets or resuscitative endovascular balloon occlusion of the aorta (REBOA). As used herein, ALI is defined to include the loss of perfusion to an extremity from any cause, including a loss of blood flow due to the application of a tourniquet. In four years of recent war in Iraq and Afghanistan, limb threatening extremity injuries cost the military $40 million in immediate costs plus $1 billion in lifetime disability costs. Every year in the United States, ALI affects 200,000 people and leads to 40,000 amputated arms and legs at a cost to the healthcare system of $7 billion, as well as a human cost impossible to quantify.

Therapeutic cooling has been shown to prevent ischemic damage in the laboratory and in organ transplantation, neurologic surgery, and cardiac surgery by reducing tissue and organ metabolic rate. Cooling is used sporadically by surgeons to preserve ischemic limbs in the field and during transport but this has not become standard of care due to logistical difficulties in providing sustained cooling to a limb in situ. A therapeutic limb cooling system is disclosed as a solution to potentially rescue life and limb in traumatic acute limb ischemia. It is believed that, in a swine model, experimental limb cooling to 5-15° C. would improve local limb muscle tissue metabolism during ALI compared to uncooled limbs.

Joint Theater Trauma Registry data from 2006-2011 revealed that 14% of casualties with major vascular injury who underwent damage control resuscitation (DCR) and vascular repair still required delayed amputation. These casualties were evacuated quickly with fasciotomy and vascular shunting to decrease ischemic limb injury. Fasciotomy, rapid transport, and temporary shunting are not adequate to prevent amputation in casualties with ALI receiving DCR. Further, a large number of patients go on to suffer permanent disability from muscle and nerve injury despite successful limb salvage.

A rise in asymmetric warfare and terrorist attacks in military settings, as well as civilian (accidental) trauma-inducing events have led directly to an increase in the occurrence of ALI. Tourniquet use has experienced a resurgence following recent military experience that demonstrates that tourniquets save lives. This practice has reached the policy level of mandating tourniquet availability and training in the civilian setting. After the Sandy Hook school shooting, the Hartford Consensus was formed and the Executive Branch and FEMA enacted policy to promote hemorrhage control training among the public including, specifically, tourniquet use. These policies are reaching the state level. However, limbs are still being lost, perhaps as a consequence of tourniquet use itself. The possibility of extending the time which a tourniquet can be used safely is an unmet need of trauma, military, and pre-hospital medicine. Similarly, REBOA is gaining popularity in the civilian and military settings. However, the primary complication of REBOA use is ALI, with the rate of limb loss associated with REBOA being as high as 12% in some series. Further, in addition to traumatic and iatrogenic causes of ALI, atherosclerosis is the primary cause of ALI overall and is a chronic disease of aging. With the rapid surge in our elderly civilian population—termed by some as the ‘silver tsunami’—we can expect a continuing increase in non-traumatic ALI cases and limb loss.

Recent studies, including animal studies, suggest that the period prior to nerve and other tissue damage can be extended, and/or nerve damage can be minimized, if the limb and, in particular, the tissue region including the nerve is rapidly and significantly cooled. Because of the low thermal conductivities of human tissue, however, rapid core limb cooling can be difficult to achieve. To provide core (deep) limb cooling rapidly it is necessary to lower skin temperatures about the ischemic limb. However, subzero skin temperatures can lead to tissue injury and destruction, most notably frostbite or cold burn. Moreover, at sufficiently cold surface temperatures, typically near or below 0° C., blood vessels close to the skin will constrict. Therefore, the body's own protective mechanisms may hinder rapid core limb cooling.

The present inventor has obtained animal study data suggesting that external cooling to 5-15° C. favorably affects limb metabolism during acute limb ischemia in a controlled pig model. Armed with this data and using computer heat transfer models, a novel tourniquet device has been developed that may be used to provide therapeutic cooling to human lower limbs in the event of ischemia. The ischemia may be induced by the device itself, for example, to preserve life in traumatic injury scenarios, or may result from other causes such as a blood clot, or the like. This foundational work will enable rapid translation of limb cooling as a viable new approach to limb salvage during ALI.

As noted earlier, ALI affects approximately 200,000 people in the United States annually, leading to approximately 40,000 amputated arms and legs. When ALI occurs, rapid transfer to a tertiary care facility and restoration of blood flow to the extremity by a vascular surgeon is the only treatment. Every minute without blood flow increases the likelihood of amputation or death. Therefore rapid surgical revascularization is imperative to avoid permanent tissue damage and loss of limb. Improvements to ALI individual outcomes may be improved if ischemic tissue damage, and in particular nerve damage, can be delayed or reduced prior to revascularization.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

A cooling tourniquet system includes a cooling component having a cooling pad and a cover member. The cooling pad is configured to be wrapped about a limb of a body with a contact surface for placement in contact with the limb. The cover member encloses the cooling pad and includes a tourniquet on a proximal portion of the cover member. The temperature of the contact surface is controllable during use, and includes one or more temperature sensors that are configured to detect a temperature of the limb and to generate corresponding limb temperature data. A control component is operably connected to receive the limb temperature data from the at least one temperature sensor, and configured to control the temperature of the contact surface based on the received limb temperature data. For example, the control component may control the temperature of the contact surface to achieve a rapid deep cooling of the limb of the body without causing any frostbite or other cold-related tissue damage.

In an embodiment the control component generates a lower contact surface temperature when the received limb temperature data indicates the limb is relatively warm, and to produce increasing contact surface temperature as the limb temperature data indicates the limb is cooling.

In an embodiment the cooling pad includes a fluid bladder having an inlet port and an outlet port, and the control component is configured to control the temperature of a coolant fluid flowing into the inlet port. In some embodiments the control component also or alternatively controls a flow rate of the coolant fluid flowing into the inlet port.

In an embodiment the control component includes a pump that is controlled by the processor to modulate the flow rate of the coolant fluid to the inlet port.

In some embodiments the control component includes a chiller controlled by the processor, for controlling the temperature of the coolant fluid.

In some embodiments the control component includes a reservoir of coolant fluid, a chiller configured to control a temperature of the coolant fluid, and a pump for pumping coolant fluid from the reservoir to the inlet port.

In some embodiments the control component includes a display controlled by the processor to display a limb temperature based on the temperature data.

In some embodiments a plurality of temperature sensors are provided and spaced apart on the contact surface of the cooling pad.

In some embodiments the cooling pad has a plurality of zones, and the control component separately controls a respective contact surface temperature of each of the plurality of zones.

In some embodiments a distal portion of the cover member has an air bladder configured to urge the cooling pad against the limb during use.

In some embodiments the cover member includes a display operatively connected to the control component and configured to display a status of the cooling tourniquet system.

A method of treating acute limb ischemia in a limb includes wrapping a cooling component about the limb with a contact surface of the cooling component in contact with the limb, wherein the cooling component comprises (i) a cooling pad positioned against the limb, (ii) at least one sensor positioned to detect a temperature of the limb adjacent the cooling pad, and (iii) a cover member supporting the cooling pad against the limb, wherein the cover member further comprises a flexible proximal portion, and a tourniquet supported on the proximal portion and configured to be selectively applied to stop blood flow to the limb distally from the tourniquet. The method includes controlling a temperature of the cooling pad based on temperature data from the at least one sensor to produce a time-varying temperature to the limb to accelerate the cooling of the limb without causing damage to limb tissue.

In an embodiment the sensor(s) is in contact with the limb, and the cooling pad is controlled by a control component that includes a processor operably connected to receive temperature data from the at least one sensor, and to use the received temperature data to control the temperature of the cooling pad.

In an embodiment, if the limb has a traumatic injury resulting in a life-threatening loss of blood the tourniquet is applied at a proximal location on the limb.

In an embodiment the control component produces a time-varying temperature of the cooling pad to achieve a rapid deep cooling of the limb.

In an embodiment the cooling pad is a fluid bladder having an inlet port and an outlet port, and the control component is configured to control a temperature of coolant fluid flowing into the inlet port.

In an embodiment the control component controls a flow rate of coolant fluid flowing into the inlet port, for example with a pump controlled by the processor to modulate the flow rate, and further controls a chiller to control the coolant fluid temperature.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a cooling sleeve and tourniquet in accordance with the present invention, shown for application to a human leg;

FIG. 2 illustrates an exploded view of the cooling sleeve and tourniquet shown in FIG. 1;

FIG. 3 illustrates a liquid bladder assembly for the cooling sleeve and tourniquet shown in FIG. 1;

FIG. 4 illustrates a cover with an integrated tourniquet for the cooling sleeve and tourniquet shown in FIG. 1; and

FIG. 5 is a system diagram for the cooling sleeve and tourniquet shown in FIG. 1.

### DETAILED DESCRIPTION

Although acute limb ischemia (ALI) is a treatable condition, delayed treatment can result in permanent disability, amputation, or even death to the victim. The standard of care for ALI is surgical intervention. The inventors are unaware of any pre-surgical intervention options to improve outcomes related to ALI. As discussed above, in some instances ALI may be induced to prevent loss of life or limb. For example, in the event of an injury to a limb causing a rapid loss of blood, a tourniquet may be applied proximally from the injury to control venous and arterial circulation to the injured limb for a period of time. Sufficient pressure is applied to cause the underlying vessels to occlude. For example, tourniquets are frequently required for in-field treatment of injuries to military personnel.

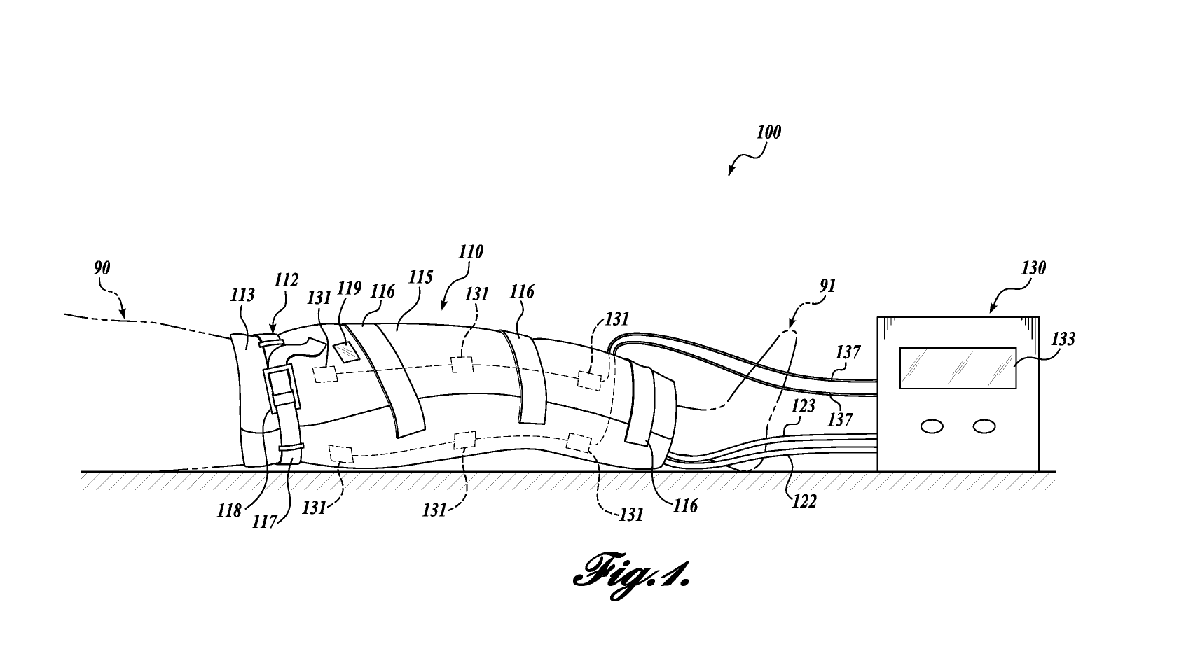

A novel tourniquet system is disclosed that prevents or reduces tissue damage from ALI, whether the ALI results from internal vessel blockages or from the application of a tourniquet to prevent life-threatening loss of blood. An embodiment of a cooling sleeve and tourniquet system 100 in accordance with the present invention is shown in FIG. 1 applied to a leg or limb 91 of an individual 90. For example, the tourniquet system 100 may be used to treat soldier or civilian victims of an explosive device in the field prior to transporting the victim to a medical facility. The system 100 may also be used (without applying the tourniquet) for a victim of ALI resulting from a non-traumatic cause, for example an embolism, during transportation to a hospital.

The cooling device and tourniquet system 100 in this embodiment includes a multi-part cooling/tourniquet component 110 configured to be wrapped around the limb 91, and a control component 130 that monitors and controls the temperature applied by the cooling/tourniquet component 110 in order to produce rapid deep cooling. In particular, the system 100 is configured to cool the injured limb 91 at least to a depth that includes the major nerves in the limb 91 as rapidly as possible, without causing frostbite or other tissue damage to the limb 91.

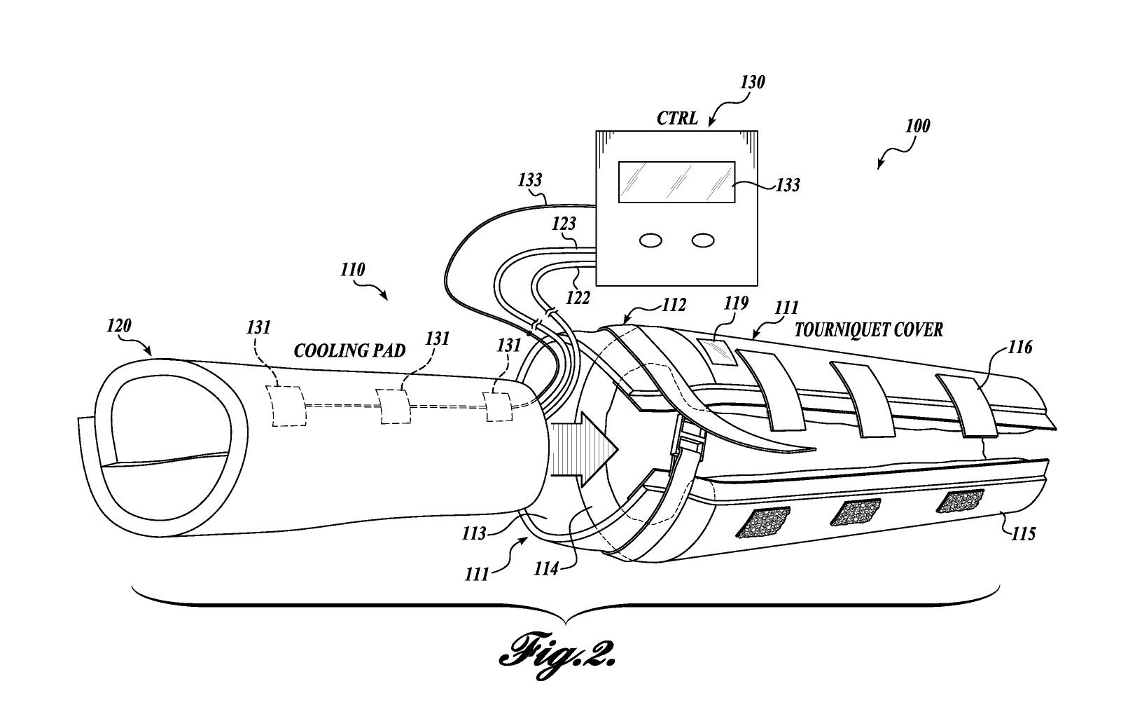

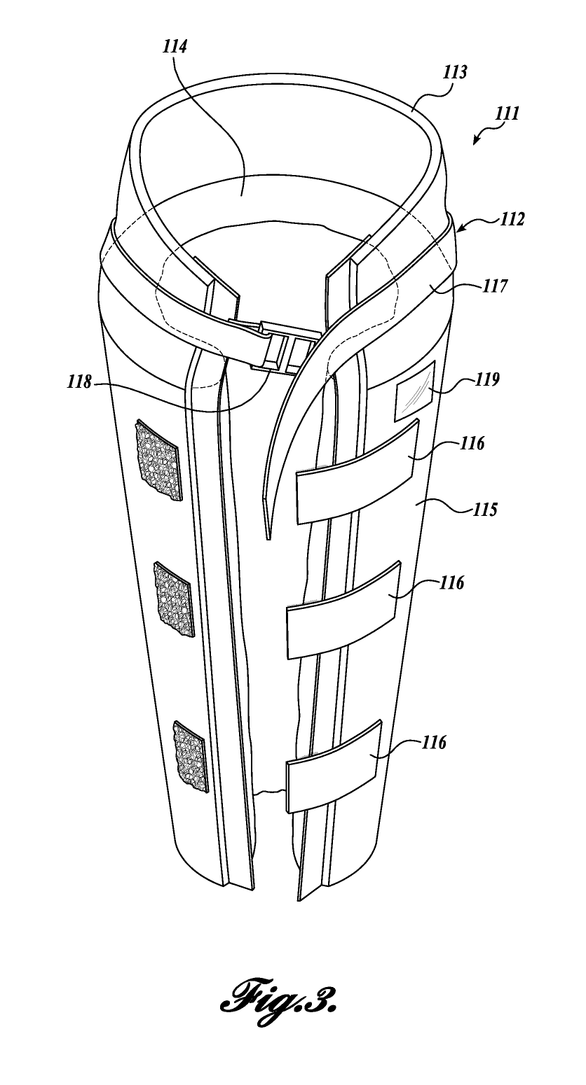

Refer now also to FIG. 2, which illustrates the cooling/tourniquet component 110 with a cover portion 111 separated from a cooling pad 120. Refer also to FIG. 3, which shows the cover portion 111 in isolation. The cover portion 111 includes a relatively thin proximal sleeve portion 113 and a relatively thick distal portion 115. The proximal sleeve portion 113 includes an integrated tourniquet 112, for example, a flexible strap 117 and an adjustable securement mechanism 118. Although a manual tourniquet 112 is illustrated, other types of tourniquets are contemplated and may alternatively be used. For example, a pneumatic tourniquet may be used.

The tourniquet 112 is configured to be tightened about the limb 91 with sufficient pressure to occlude blood flow to the limb 91, and to maintain the pressure until loosened by the caregiver. The distal portion 115 includes an inner support device, for example, an air bladder 114. The support device 114 is configured to provide a supporting pressure to the limb 91, pressing the cooling pad 120 against the limb 91 during use. In some embodiments the bladder 114 is operatively connected to an inflator (not shown) such that the bladder 114 may be inflated to a desired pressure after securing the component 110 to the limb. The bladder 114 also provides the benefit of essentially immobilizing or stabilizing the limb 91. Other methods of stabilizing the limb may be used. In some embodiments the bladder 114 may be provided external of the cover portion 111. Alternatively or additionally, a system of relatively rigid bars in an insulating foam may be used. Optionally, the cover portion 111 may include a display 119, to display data regarding the system 100. For example, the display 119 may display a skin temperature of the limb 91, and/or a time since the system 100 was engaged, or the like.

A plurality of securement straps 116 close the cover portion 111 and secure the cooling pad 120 around the limb 91. Other securement mechanisms are known and may be used. For example, the cover portion 111 may be provided with a zipper or hook-and-loop fastening means along a length of the cover 111 for closing the cover portion 111 about the limb 91.

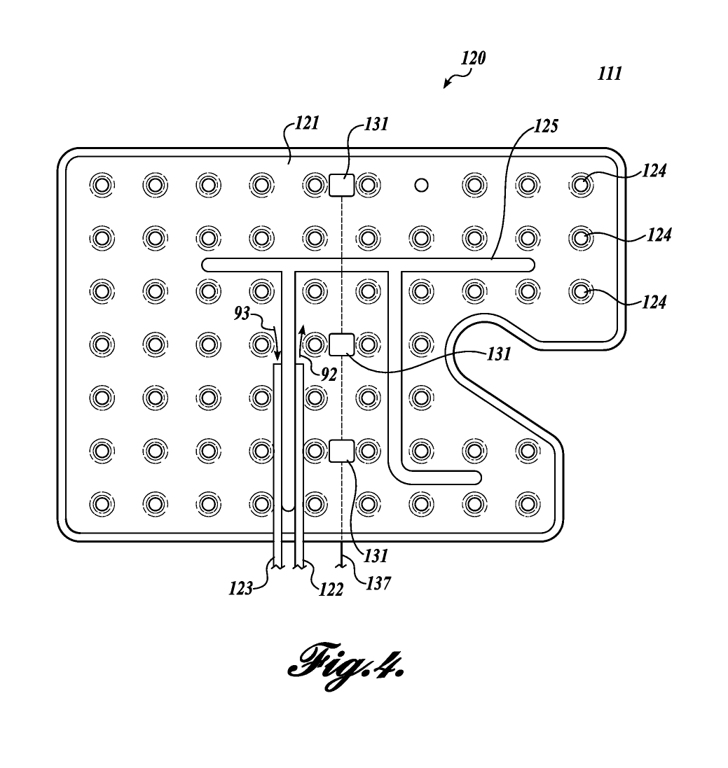

Refer now also to FIG. 4, which is a plan view of the cooling pad 120. The cooling pad 120 in this embodiment comprises a fluid bladder 121 having an inlet port 122 and an outlet port 123. An array of attachment points 124 (for example, heat welds) connect opposite sides of the fluid bladder 121 such that opposite walls of the fluid bladder 121 are joined, thereby limiting the thickness of the bladder 121 under pressure. Elongate heat welds 125 provide flow blockages in the fluid bladder 121, to define a general flow path through the fluid bladder 121 between the inlet port 122 and the outlet port 123. For example, in this embodiment fluid entering through the inlet port 122 (indicated by arrow 92) must travel around the elongate heat welds 125 to return to the outlet port 123 (indicated by arrow 93). A plurality of temperature sensors 131 are fixed to an outer surface of the cooling pad 120, and positioned to engage the limb 91 when the system 100 is in use. A means for transmitting the data from the sensors 131 is provided, for example, a wireless system, a signal wire 137, or the like.

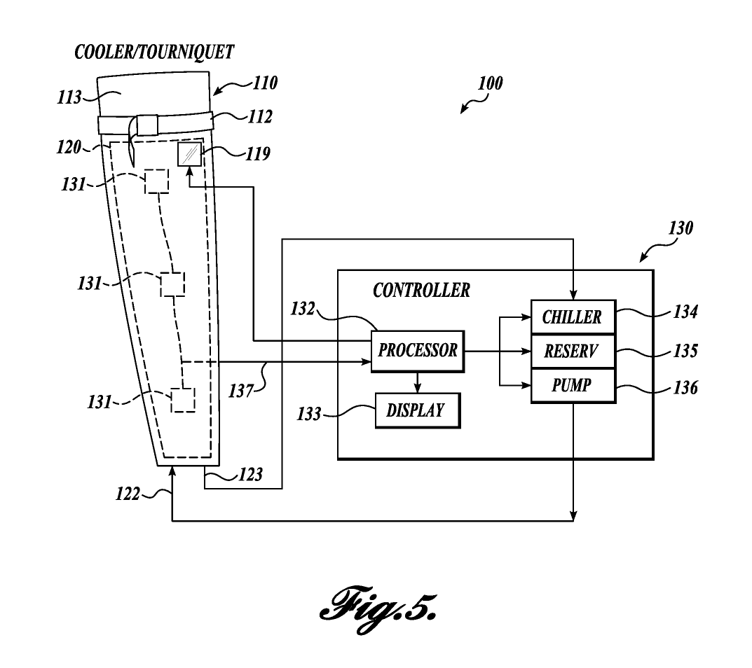

Refer now to FIGS. 1 and 5. FIG. 5 illustrates in diagram the system 100, including components of the control component 130. The controller component 130 includes a processor 132 that is in signal communication with one or more sensors 131, for example, temperature sensors configured to detect a temperature at or near the skin of the limb 91, and/or pressure sensors configured to detect a pressure in the fluid bladder 121 or in the air bladder 114. The processor 132 may optionally drive a display 133 that displays information regarding the status of the system 100. For example, the display 133 may be configured to display one or more of a temperature of the limb 91, a temperature of the cooling fluid, a flow rate of the cooling fluid, a time showing how long the system 100 has been in continuous operation, a countdown timer, a pressure of the bladders 114, 121, or the like. Additionally or alternatively, the processor 132 may control the display 119 (if present) on the cooling/tourniquet component 110.

The control system 130 in this embodiment further includes a fluid chiller 134, a coolant reservoir 135, and a pump 136. The processor 132 receives data from the sensors 131, and uses the received data to operate the chiller 134 and the pump 136, to provide a controllable coolant flow rate and coolant flow temperature to the inlet port 122 of the cooling pad 120. Optionally, the control system 130 may also be operatively connected 138 to the display 119 on the cover portion 111, for example, to display data regarding the status of the system 100.

For example, in order to cool the injured limb 91 as rapidly as possible, the control system 130 may initially produce a cooling fluid flow through the cooling pad 120 at a relatively high flow rate and/or at a relatively low temperature. For example, the coolant temperature may initially be below zero degrees Celsius. As the temperature of the injured limb 91 decreases (based on data from the sensors 131, the flow rate and/or the cooling fluid temperature may be moderated, for example to between 5° C. and 15° C., to avoid causing tissue damage. It will also be appreciated that the inner support device 114 of the cooling component 120 (for example, the air bladder) urges the cooling pad 120 firmly against the limb 91, to ensure good thermal contact is maintained to facilitate heat transfer, and to provide accurate sensor data to the controller component 130.

As discussed above, in order to prevent or moderate tissue damage, and in particular nerve damage, is it desirable to provide deep cooling of the limb 91 as rapidly as possible.

To use the system 100 the cooling pad 120 is placed around the limb, with a surface of the cooling pad in contact with the limb. It is contemplated that a thin fabric (not shown), for example a thin cotton fabric wrap or sleeve, may be provided between the limb and the contact surface of the cooling pad 120. Such an arrangement is expressly defined to be encompassed by the contact surface of the cooling pad being in contact with, or adjacent, the limb. In some embodiments the thin wrap or sleeve may be wet, for example by soaking in water. The cooling pad 120 sensors 131 detect a temperature of the limb adjacent the cooling pad 120. The cover portion 111 supports the cooling pad 120 against the limb. If indicated by the situation, for example if a significant loss of blood is occurring, the tourniquet 112 on the relatively flexible portion of the cover portion 111 is tensioned to stop the distal flow of blood. The control component 130 controls the temperature of the cooling pad 120, e.g., by controlling the fluid temperature to the cooling pad 120, based on temperature data from the sensors 131, to produce a time-varying temperature to the limb to accelerate the cooling of the limb without causing damage to limb tissue.

Although a currently preferred embodiment of the cooling and tourniquet system has been described, other embodiments are contemplated. For example, in another embodiment an array of thermoelectric cooling (TEC) elements are provided in a flexible wrap that is applied to the injured limb 91. The TEC elements are controlled by the control component, based on sensor feedback from the injured limb 91, to provide controlled cooling to limb 91. It will be appreciated that the array of TEC elements provide the advantage that the cooling may be more precisely controlled. In particular, the TEC elements may be individually controlled, so that the cooling temperature applied to the limb may vary along the surface of the limb.

Similarly, it is contemplated that the cooling pad 120 may have a plurality of independently controlled sections. For example, the cooling pad may have two or more fluidly separated sections, each with separate inlet and outlet ports, and the control component may be configured to provide flows to each section based on sensor data received from the portion of the limb corresponding to each section, such that the cooling is provided zonally.

While illustrative embodiments have been illustrated and described, it will be appreciated that various changes can be made therein without departing from the spirit and scope of the invention.

The embodiments of the invention in which an exclusive property or privilege is claimed are defined as follows:

1. A cooling tourniquet system comprising:
a cooling component having a cooling pad configured to be wrapped about a limb of a body with a contact surface for placement in contact with the limb, and a cover member configured to enclose the cooling pad wrapped about the limb, wherein a temperature of the contact surface is controllable during use, and further comprising at least one temperature sensor on the contact surface that is configured to detect a temperature of the limb and to generate corresponding limb temperature data;
wherein the cover member further comprises a proximal portion, a distal portion, and a tourniquet supported on the proximal portion, the tourniquet configured to be selectively applied to stop blood flow to the limb distally from the tourniquet;
a control component comprising a processor operably connected to receive the limb temperature data from the at least one temperature sensor, and configured to control the temperature of the contact surface based on the received limb temperature data.

2. The cooling tourniquet system of claim 1, wherein the control component is configured to control the temperature of the contact surface to achieve a rapid deep cooling of the limb.

3. The cooling tourniquet system of claim 2, wherein the control component is configured to produce a lower temperature of the contact surface when the received limb temperature data indicates the limb is relatively warm, and to produce an increasing temperature of the contact surface as the limb temperature data indicates the limb is cooling.

4. The cooling tourniquet system of claim 1, wherein the cooling pad comprises a fluid bladder having an inlet port and an outlet port, and the control component is configured to control a temperature of a coolant fluid flowing into the inlet port.

5. The cooling tourniquet system of claim 4, wherein the control component is further configured to control a flow rate of the coolant fluid flowing into the inlet port.

6. The cooling tourniquet system of claim 4, wherein the control component further comprises a pump that is controlled by the processor to modulate a flow rate of the coolant fluid to the inlet port.

7. The cooling tourniquet system of claim 4, wherein the control component further comprising a chiller controlled by the processor, wherein the chiller is configured to control the temperature of the coolant fluid.

8. The cooling tourniquet system of claim 4, wherein the control component further comprises a reservoir of the coolant fluid, a chiller configured to control the temperature of the coolant fluid in the reservoir, and a pump configured to pump the coolant fluid from the reservoir to the inlet port.

9. The cooling tourniquet system of claim 4, wherein the control component further comprises a display controlled by the processor to display a temperature based on the received limb temperature data.

10. The cooling tourniquet system of claim 1, wherein the at least one temperature sensor on the contact surface of the cooling pad comprises a plurality of spaced-apart temperature sensors disposed on the contact surface of the cooling pad.

11. The cooling tourniquet system of claim 1, wherein the cooling pad comprises a plurality of zones, wherein the control component is configured to control a respective contact surface temperature of each of the plurality of zones.

12. The cooling tourniquet system of claim 1, wherein the distal portion of the cover member further comprises a bladder configured to urge the cooling pad against the limb.

13. The cooling tourniquet system of claim 1, wherein the cover member further comprises a display operatively connected to the control component and configured to display a status of the cooling tourniquet system.

14. A method of treating acute limb ischemia in a limb, the method comprising:
wrapping a cooling component about the limb with a contact surface of the cooling component in contact with the limb, wherein the cooling component comprises (i) a cooling pad positioned against the limb, (ii) at least one sensor positioned to detect a temperature of the limb adjacent the cooling pad, and (iii) a cover member supporting the cooling pad against the limb, wherein the cover member further comprises a flexible proximal portion, and a tourniquet supported on the proximal portion and configured to be selectively applied to stop blood flow to the limb distally from the tourniquet;
controlling a temperature of the cooling pad based on temperature data from the at least one sensor to produce a time-varying temperature to the limb to accelerate the cooling of the limb without causing damage to limb tissue.

15. The method of claim 14, wherein the at least one sensor is in contact with the limb, and wherein the cooling pad is controlled by a control component comprising a processor operably connected to receive temperature data from the at least one sensor, and configured to control the temperature of the cooling pad based on the received temperature data.

16. The method of claim 14, further comprising the step of determining if the limb has a traumatic injury resulting in a life-threatening loss of blood and, if the traumatic injury is present, applying the tourniquet.

17. The method of claim 15, wherein the control component is configured to produce a time-varying temperature of the cooling pad to achieve a rapid deep cooling of the limb.

18. The method of claim 15, wherein the cooling pad comprises a fluid bladder having an inlet port and an outlet port, and the control component is configured to control a temperature of coolant fluid flowing into the inlet port.

19. The method of claim 18, wherein the control component is further configured to control a flow rate of coolant fluid flowing into the inlet port.

20. The method of claim 18, wherein the control component further comprises a pump controlled by the processor to modulate the flow rate of coolant fluid to the inlet port, and a chiller controlled by the processor, wherein the chiller is configured to control a coolant fluid temperature.

21. The method of claim 18, wherein the at least one sensor comprises a plurality of spaced-apart temperature sensors.
